(19) United States
(12) Patent Application Publication
(10) Pub. No.: US 2025/0229365 A1
(43) Pub. Date: Jul. 17, 2025

(54) MODELING METHOD AND SYSTEM OF CRYSTAL PLASTICITY FINITE ELEMENT MODEL FOR WELD MICROSTRUCTURE CRYSTAL PLASTICITY

(71) Applicant: Huazhong University of Science and Technology, Wuhan (CN)

(72) Inventors: Shaoning GENG, Wuhan (CN); Ping JIANG, Wuhan (CN); Minjie SONG, Wuhan (CN); Leshi SHU, Wuhan (CN)

(73) Assignee: Huazhong University of Science and Technology, Wuhan (CN)

(21) Appl. No.: 19/023,632

(22) Filed: Jan. 16, 2025

(30) Foreign Application Priority Data
Jan. 16, 2024 (CN) 2024100609564

(51) Int. Cl. B23K 31/12; B23K 26/21; G06F 30/23; G06F 111/10; G06F 119/10

(52) U.S. Cl. CPC B23K 31/12; G06F 30/23; B23K 26/21; G06F 2111/10; G06F 2119/10

(57) ABSTRACT

The invention relates to the technical field of metal material processing engineering, discloses a full-automatic full-process modeling method and system of crystal plasticity finite element model for laser welding weld, which adopts MATLAB to call ABAQUS and PYTHON for co-simulation programming. In this text, a full-automatic modeling method of the crystal plasticity finite element model for laser welding weld is proposed, and a one-key crystal plastic finite element model for laser welding weld is established based on EBSD experimental data. In addition, the full-process full-automatic modeling method of crystal plastic finite element for laser welding weld is universal, and can be used for the establishment of the crystal plasticity finite element model for the microstructure of the base material of aluminum alloy, titanium alloy, magnesium alloy, stainless steel and other materials.

### CROSS-REFERENCE TO RELATED APPLICATIONS

The application claims priority to Chinese patent application No. 202410060956.4, filed on Jan. 16, 2024, the entire contents of which are incorporated herein by reference.

### TECHNICAL FIELD

The present invention relates to the technical field of metal material processing engineering, in particular to a full-automatic full-process modeling method of the crystal plasticity finite element model for laser welding weld microstructure.

### BACKGROUND

Laser welding is an important process in the manufacture of key components of metal materials, but the laser welded joint of materials is also an area prone to failure behavior, the main reason is that under the action of laser welding thermal cycle, the welding area occurs melting and recoagulation, resulting in the dissolution and evaporation of the strengthened phase, the deterioration of microstructure, and the decline of mechanical properties.

The crystal plastic finite element method combines the crystal plastic theory and the finite element analysis method, which can accurately describe the behavior of materials on the microscopic scale (deformation behavior, failure behavior), and is an important technology to reveal the evolution law of mechanical properties and failure mechanism of materials. However, the microstructure of the material after laser welding changes greatly compared with that of the base material. The traditional methods of establishing the crystal plasticity finite element model (such as Neper modeling, Voronoi modeling, METX modeling, and the like) used to establish the crystal plasticity finite element model of laser welding weld have disadvantages such as a low accuracy, a low efficiency and a complex flow. Therefore, a full-automatic, full-process, high-precision and high-efficiency finite element modeling method for laser welding weld crystal plasticity is needed to solve the difficult problem of laser welding weld crystal plasticity finite element modeling.

Through the above analysis, the existing problems and defects of the existing technology are as follows:

The microstructure of the material after laser welding changes greatly compared with that of the base material. The traditional methods of establishing the crystal plasticity finite element model (such as Neper modeling, Voronoi modeling, METX modeling, and the like) used to establish the crystal plasticity finite element model for laser welding weld have disadvantages such as a low accuracy, a low efficiency and a complex flow.

### SUMMARY

With respect to the problems in the existing technology, the present invention provides a full-automatic full-process modeling method of the crystal plasticity finite element model for laser welding weld.

The invention is realized in this way, a full-automatic full-process modeling method of the crystal plasticity finite element model for laser welding weld includes:
Step 1: using MATLAB to call METX toolbox to import EBSD data of laser welding welds, and creating EBSD data set in MATLAB workspace;
Step 2: using grain segmentation, confidence index filtering, small grain filtering and other methods to establish a grain set of original EBSD data, removing the grain in a very small size, and reducing a noise of the original laser weld EBSD data;
Step 3: extracting the EBSD data after noise reduction, including the grain number, equivalent diameter, Euler angle of grain orientation, grain pixel coordinates, grain set and other data of each grain, drawing a grain distribution map after noise reduction, and comparing with the original laser welding weld EBSD grain distribution map;
Step 4: extracting a length and a width of the original EBSD scan data through MATLAB, calculating a step size of a single pixel of the EBSD scan data of laser welding welds, and converting an unit from um to mm;
Step 5: entering a mesh size of the crystal plasticity finite element model of laser welding weld, an initial analysis step length, the minimum analysis step length and the maximum analysis step length in the ABAQUS analysis step;
Step 6: writing py files to generate and create an initial model of ABAQUS through MATLAB; executing the py file by calling PYTHON through MATLAB to automatically generate the INP file of the initial model;
Step 7: reading each node data and unit data of the initial model by MATLAB, and generating a data file of the node and an unit set of each grain in ABAQUS according to a location of each grain and a pixel value occupied by each grain, and writing into the INP file of the initial model, and generating the INP file of the crystal plasticity finite element model, including the grain structure distribution of laser welding weld.
Step 8: calling ABAQUS GUI interface in MATLAB to import INP file to verify a consistency of crystal plasticity finite element model with grain structure distribution of laser welding weld and original laser welding weld microstructure distribution;
Step 9: using MATLAB to call METX toolbox to draw a polar figure of the EBSD data of the original laser welding weld and a polar figure of the grain set after noise reduction, and verifying whether the grain set established is consistent with the original EBSD data;
Step 10: converting a grain Euler Angle into radian value, and using MATLAB to output a PYTHON file that assigns material properties to each grain, wherein the material property assigning code includes three main parts: automatically reading the grain Euler Angle radian value, converting radian value to Miller index, and assigning a grain size effect;
Step 11: using MATLAB to call ABAQUS to perform PYTHON for material property assignment, assigning material properties to each grain, and generating a final INP file for the crystal plasticity finite element model of the laser welding weld;
Step 12: generating an empty dat file through MATLAB to write the orientation information of each grain in the final crystal plasticity finite element model output by ABAQUS;
Step 13: calling ABAQUS through MATLAB to run a final generated INP file, and calling a subroutine of crystal plasticity finite element simulation to output the orientation information of each grain in the final generated crystal plasticity finite element model; calling METX to draw the polar figure of each grain in the final output, and comparing it with the polar figure generated in step 8;
Step 14: writing a PYTHON file to extract a stress-strain curve of crystal plasticity finite element simulation through MATLAB, and calling ABAQUS to execute the PYTHON file through MATLAB to obtain the stress-strain curve of crystal plasticity finite element simulation.

Further, the data set includes important information such as a grain type, a grain ID, a grain orientation, a phase sequence, and a grain rotation.

Further, the formula of the grain segmentation, the confidence index filtering and the small grain filtering is as follows:

$$Δ θ = arccos ( \frac{trace ( {R}_{1}^{T} {R}_{2} ) - 1}{2} ) ;$$

wherein, R₁^{T} and R₂ respectively represent a rotation matrix of grains, and Δθ are an orientation angle difference between grains; if Δθ is greater than a threshold, the two grains are considered to belong to different grains;

$$f ( {CI}_{i} ) = \{ \begin{matrix}1 , & if {CI}_{i} > min Ci \\ 0 , & otherwise\end{matrix} ;$$

For each measurement point i, the confidence index is CIᵢ;

$$f ( g ) = \{ \begin{matrix}1 , & if size ( g ) > min Size \\ 0 , & otherwise\end{matrix} ;$$

wherein g is each grain and size(g) is a size of each grain.

Further, the py file includes INP files for creating model entities, meshing, generating meshing elements, creating assemblies, establishing analysis steps, creating reference points, establishing reference point coupling constraints, adding fixed constraints, adding load constraints and generating model.

Further, the formulas for converting the radian value to Miller index and grain size effect are as follows:

$$( \begin{matrix}u & r & h \\ v & s & k \\ w & t & l\end{matrix} ) = [ \text{} \begin{matrix}\begin{matrix}cos ψ cos φ - \\ sin ψ sin φ cos θ\end{matrix} & \begin{matrix}sin ψ cos θ + \\ cos ψ sin φ cos θ\end{matrix} & sin φ sin θ \\ \begin{matrix}- cos ψ sin φ - \\ sin ψ cos φ cos θ\end{matrix} & \begin{matrix}- sin ψ sin φ + \\ cos ψ cos φ cos θ\end{matrix} & cos φ sin θ \\ sin ψ sin θ & - cos ψ sin θ & cos θ\end{matrix} ] ;$$

$${τ}_{i} = {τ}_{0} + {Kd}^{- 0.5} ( i = 1 , 2 , 3 … ) ;$$

wherein θ is the nutation Angle, ψ is the precession Angle, and φ is the rotation Angle; u, v, w are crystal direction indices; h, k, l are crystal face indices; i is the grain number, T0 is the initial yield strength of the grain, K is the influence coefficient of the reaction grain boundary on the deformation, and d is the equivalent diameter of the grain.

Another objective of the invention is to provide an automatic full-process modeling system of the crystal plasticity finite element model for laser welding weld, which includes the following:
data processing module, equipped with MATLAB software and METX toolbox, used to import and process EBSD data of laser welding welds, including grain segmentation, confidence index filtering and small grain filtering;
model building module, using MATLAB to extract and process data, including calculating the individual pixel step size of EBSD scanning data, converting units, and generating a grain distribution map;
finite element analysis module, using ABAQUS software and data generated by MATLAB, used to create an initial model, a grain node and a unit collection data file, and generate a crystal plasticity finite element model of laser welding weld microstructure characteristics;
model verification module, using ABAQUS GUI and MATLAB combined with METX toolbox, used to import models, verify a consistency of models, draw and compare polar maps;
material property assigning module, calling ABAQUS through MATLAB to execute Python scripts, assigns material properties to each grain, and generates the final finite element model INP file;
data output module, used to generate and process ABAQUS output data, including orientation information and stress-strain curves for each grain in the crystal plasticity finite element model.

Combined with the above technical solution and solved technical problems, the technical solution to be protected by the invention has advantages and positive effects as follows:

Firstly, in view of the technical problems existing in the above existing technology and the difficulty of solving the problem, some creative technical effects are brought about after solving the problem. The specific description is as follows:
by using MATLAB to call ABAQUS and PYTHON for co-simulation programming, the invention innovatively proposes a full-process full-automatic modeling method of the crystal plasticity finite element model for laser welding weld. This method solves a problem of inefficiency and complexity in the traditional modeling process, which is embodied in the following aspects:
1. One-key generative modeling process: the invention realizes a one-key generative modeling process based on EBSD (electronic backscatter diffraction) experimental data. This process not only greatly improves an efficiency of model building, but also ensures an accuracy and repeatability of the modeling process.
2. Universality of the model: the proposed modeling method is not limited to specific materials, and its universality is reflected in the base materials and welds that can be applied to aluminum alloy, titanium alloy, magnesium alloy, stainless steel and other materials. This is of great significance to the research in the field of materials science and engineering, especially in the research and optimization of multi-material welding processes.
3. Creative technical effect: the technical problem solved by the invention is not limited to the modeling efficiency and universality, but also includes the accurate simulation of the crystal plasticity behavior of the weld microstructure. This high-precision simulation provides an important tool for understanding and optimizing microstructure changes during welding, helping to improve the performance and reliability of welded joints.
4. Result and data analysis in the research and development process: by comparing the experimental data and simulation results in the research and development process, the method of the invention shows an excellent simulation accuracy and reliability. These results not only validate the effectiveness of the method, but also provide solid data support for future research and application.

In summary, the technical solution of the invention not only solves some key problems in the existing technology, but also brings creative technical effects, and provides new ideas and tools for research and practical application in the field of laser welding.

Secondly, the invention also has the following significant technical progress:
1. Overall performance improvement: the technical solution of the invention integrates the functions of MATLAB, ABAQUS and PYTHON in the field of laser welding, and creates a new modeling method of the crystal plasticity finite element model for laser welding weld. This integration not only improves the efficiency of modeling, but also significantly improves the accuracy and reliability of the model, providing a strong technical support for the development of laser welding technology.
2. Innovative one-key generative modeling process: the invention greatly simplifies the traditional modeling step through the one-key generative modeling process based on EBSD experimental data. This simplification not only reduces the complexity of modeling, but also makes the creation of models faster and more efficient, which is of great significance for accelerating the development cycle and reducing costs.
3. Wide material applicability: compared with the traditional finite element modeling method, the method of the invention has significant advantages in material applicability. It can be applied to aluminum alloy, titanium alloy, magnesium alloy, stainless steel and other engineering materials, which makes the method has a high practical value in a variety of different industrial applications.
4. Improve product quality and performance: by accurately simulating the crystal plasticity behavior of the weld microstructure, the invention can help engineers better understand and control the welding process, and thus improve the quality and performance of welding products. This is particularly important for applications that require a very high welding quality (such as aerospace, automotive manufacturing, and the like).
5. Combination of experiment and simulation: the method of the invention combines experimental data and simulation analysis to provide a deep understanding of the microstructure changes in the welding process. This combination not only improves the accuracy of the model, but also provides a scientific basis for the subsequent process optimization and material selection.

In summary, the technical solution of the invention is not only innovative in technology, but also has significant economic and social benefits in practical application, and provides a new perspective and tool for the development and application of laser welding technology.

Thirdly, the expected benefits and commercial value after the transformation of the technical scheme of the invention are as follows: the technical solution of the invention is expected to bring significant commercial value by realizing the full-process and full-automatic modeling of the crystal plasticity finite element model for the laser welding weld. First, the method can significantly improve modeling efficiency and reduce labor and time costs, which is especially attractive for enterprises that require a large amount of welding modeling. Secondly, because the technology supports a variety of materials, its market scope is wide, which can cover aerospace, automobile manufacturing, high-end equipment manufacturing and other industries. In addition, the accuracy and reliability of the technology help to improve the quality of welding, thereby enhancing the market competitiveness of the product and bringing higher economic benefits to the enterprise.

The technical solution of the invention fills the technical gap in the industry at home and abroad: under the present technical status, the full-automatic crystal plasticity finite element modeling method for laser welding proposed by the invention is innovative among similar technologies at home and abroad. This method integrates the co-simulation programming capabilities of MATLAB, ABAQUS and PYTHON, realizes the one-key generative modeling based on EBSD experimental data, and fills the gap of the existing technology in the field of efficient and full-automatic laser welding modeling.

The technical solution of the invention solves the technical problem that people have been eager to solve, but have not been successful: the invention successfully solves an important technical problem that has long existed in the field of laser welding: how to efficiently and accurately establish the crystal plasticity finite element model for weld. Traditional methods have limitations in modeling efficiency, accuracy and material applicability, but the invention effectively overcomes these obstacles through an innovative full-process, full-automatic method, providing new impetus for the development of welding technology.

The technical solution of the invention overcomes the technical bias: prior to this, there was a general view in the industry that efficient and accurate crystal plasticity finite element modeling for laser welding weld was difficult to achieve. The technical solution of the invention not only demonstrates the limitations of this view, but also, through its innovative technical method, demonstrates the feasibility of achieving efficient, accurate and universal modeling in the field of crystal plasticity finite element modeling of laser welding weld, thereby overcoming long-standing technical biases.

### BRIEF DESCRIPTION OF DRAWINGS

FIG. 1 is a flow chart of an full-automatic full-process modeling method of the crystal plasticity finite element model for weld provided by an embodiment of the invention.

FIG. 2 is a grain distribution diagram of the original microstructure of the laser welding weld of aluminum alloy provided by the embodiment of the invention.

FIG. 3 is a microstructure grain distribution diagram of the laser welding weld of aluminum alloy after noise reduction provided by the embodiment of the invention.

FIG. 4 is a crystal plasticity finite element model diagram of grain structure distribution of laser welding weld of aluminum alloy provided by the embodiment of the invention.

FIG. 5 is a pole diagram of the EBSD data of the laser welding weld of the original aluminum alloy provided by the embodiment of the invention.

FIG. 6 is a polar figure of the grain set of the laser welding weld of aluminum alloy after noise reduction treatment provided by the embodiment of the invention.

FIG. 7 is a polar figure of the output of a crystal plasticity finite element model provided by an embodiment of the invention.

FIG. 8 is a stress-strain curve extracted from the crystal plasticity finite element model provided by the embodiment of the invention.

### DETAILED DESCRIPTION OF THE EMBODIMENTS

In order to make the purpose, technical solution and advantages of the invention more clearly understood, the invention is further explained in the following embodiment. It should be understood that the specific embodiments described herein are intended only to explain the invention and are not intended to limit it.

The invention mainly improves the following problems and defects of the existing technology and realizes significant technical progress:
complexity and inaccuracy of data processing: traditional methods often rely on manual or semi-automated processes when processing laser welding weld EBSD data, which is not only inefficient, but also error-prone.
limitations of modeling: previous modeling processes were often limited to specific types of weld structures and lacked universal applicability and flexibility.
lack of accuracy and authenticity: traditional models often have difficulty accurately reflecting the real situation of weld microstructure, especially in details such as grain size and orientation.

With respect to the problems in the existing technology, the technical solution adopted by the invention is:
automatic data processing: using MATLAB and METX toolbox to automatically import and process EBSD data, including grain segmentation, confidence index filtering, and the like, which greatly improves the efficiency and accuracy of data processing.
full-process modeling method: from data import and processing to model generation and verification, the whole process is automated, which improves the efficiency and applicability of model establishment.
high-precision model generation: through accurate calculation and detailed processing of grain data, the established model can more truly reflect the microstructure of the weld.

Two specific application embodiments of the invention are:

### Example 1: Analysis of Laser Welding Welds in the Aviation Industry

In the aviation industry, laser welding technology is widely used to connect critical flight components. The microstructure of the weld has a decisive effect on the performance of the whole structure.
data collection: aircraft components are connected using laser welding technology and EBSD data is collected in the welding area.
data processing: using MATLAB and METX toolbox in the method of the invention to automatically process EBSD data, including grain segmentation and noise reduction processing.
model building: according to the processed data, MATLAB and ABAQUS software are used to automatically generate the crystal plasticity finite element model of the weld.
model verification and analysis: import the model through ABAQUS GUI interface, verify its consistency with the actual weld microstructure, and perform a stress-strain analysis.

By processing EBSD data automatically, the microstructure information of welding area can be obtained quickly and accurately. The stress and strain behavior of weld area can be analyzed in detail by finite element model simulation, which provides an important reference for the design and manufacture of flight parts.

### Example 2: Optimization of Laser Welding in the Automotive Industry

In automobile manufacturing, laser welding is used to connect various parts of the automotive body, and the quality of welding directly affects the safety and durability of the automobile.
data collection: EBSD data is collected during the laser welding of automotive body components.
data processing and grain analysis: the method of the invention is applied to automatically process EBSD data and draw a grain distribution map.
weld model generation: MATLAB and ABAQUS were used to generate the crystal plasticity finite element model of the weld according to the processed data.
model application and optimization: the ABAQUS simulation is run, the stress and strain of the weld is analyzed, the welding parameters is adjusted according to the results, and the welding process is optimized.

The automatic data processing method improves the efficiency and accuracy of data analysis and provides accurate microstructure information for the welding process. Finite element simulation helps to understand the performance of welds in an actual use, provides a scientific basis for the optimization of welding processes, and enhances the structural integrity and safety of automotive components.

The technical solution provided by the invention relates to an full-automatic full-process modeling method of the crystal plasticity finite element model for laser welding weld. Key technical features include:

### 1. Data Processing and Model Building:

Using MATLAB and METX toolbox to process EBSD data and creating grain data sets.

The EBSD data were denoised by the grain segmentation, the confidence index filtering and small the grain filtering, and the grain set is established.

The EBSD data after noise reduction is extracted, including a grain number, an equivalent diameter, an Euler Angle of grain orientation, pixel coordinates, and the like, and the grain distribution map is drawn and compared with the original data.

The pixel step size of EBSD scan data is calculated and the unit conversion is performed.

### 2. Automatic Generation of ABAQUS Model:

Writing and executing Python scripts through MATLAB to automatically generate the INP file of the initial model of ABAQUS;

Reading the node and unit data of the initial model, generating the node and unit collection data in ABAQUS according to the grain position and the pixel value, and writing it into the INP file.

### 3. Model Verification and Property Assignment:

By calling ABAQUS GUI interface in MATLAB, the INP file was imported to verify the consistency between the model and the original microstructure.

To verify the accuracy of grain sets, the original and denoised polar plots of grain sets are drawn.

The Euler Angle of the grain is converted to radian value, and the Python file that assigns the material properties to the grain is output.

The material Python script is executed to assign material properties to each grain.

### 4. Simulation Operation and Data Output:

An empty dat file is generated to write orientation information for each grain in the crystal plasticity finite element model.

The INP file is run, the grain orientation information is output in the crystal plastic finite element model, the polar figure is drawn, and is compared with the previous polar figure.

The stress-strain curves of crystal plasticity finite element simulation are extracted.

The invention combines MATLAB and ABAQUS to carry out a comprehensive method of data processing, model building, automatic generation, verification and simulation, and is especially suitable for studying and simulating the microstructure and material behavior of laser welding welds.

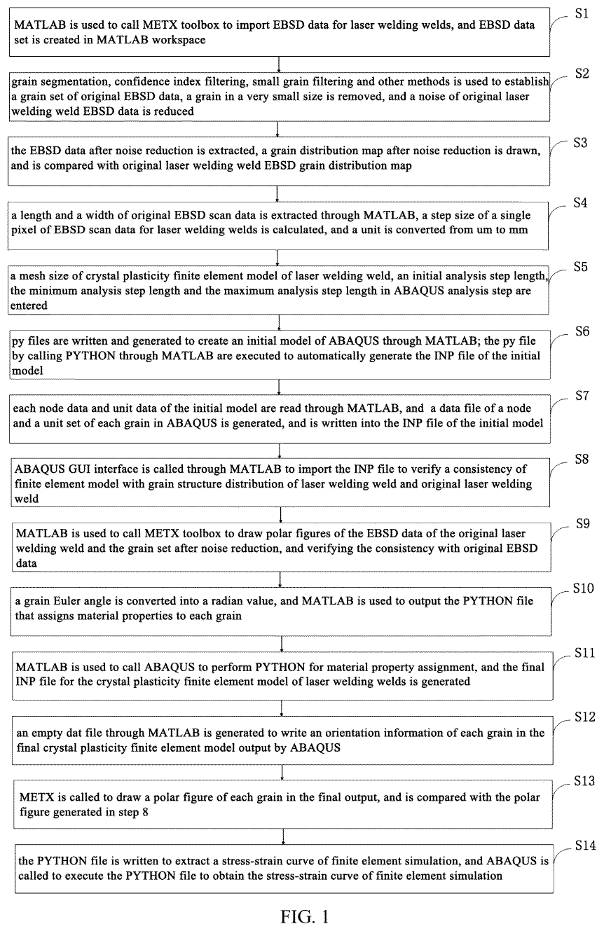

As shown in FIG. 1, the invention provides a full-automatic full-process modeling method of the finite element model for laser welding weld crystal plasticity finite element model, including the following steps:
S1, MATLAB is used to call METX toolbox to import EBSD data of laser welding welds, and EBSD data set is created in the MATLAB workspace;
S2, grain segmentation, confidence index filtering, small grain filtering and other methods are used to establish grain sets for the original EBSD data, remove the grain in a very small size, and denoise the original laser welding weld EBSD data.
S3, the EBSD data is extracted after noise reduction, including the grain number, equivalent diameter, Euler Angle of grain orientation, grain pixel coordinates, grain set and other data of each grain, the grain distribution map is drawn after noise reduction, and is compared with the EBSD grain distribution map of the original laser welding weld;
S4, the length and width of the original EBSD scan data by MATLAB is extracted, the step size of a single pixel of the EBSD scan data of laser welding welds is calculated, and the unit is converted from um to mm;
S5, the meshing size of the crystal finite element plasticity model of the laser welding weld is entered, the initial analysis step length, the minimum analysis step length and the maximum analysis step length in the ABAQUS analysis step;
S6, the py files are written and generated to create the initial model of ABAQUS through MATLAB; the py file is executed by calling PYTHON through MATLAB to automatically generate the INP file of the initial model;
S7, each node data and unit data of the initial model is read through MATLAB, data files of nodes and unit sets of each grain in ABAQUS is generated according to the location of each grain and the pixel value occupied by each grain, and are written into the INP file of the initial model, the INP file of the crystal plasticity finite element model including the grain structure distribution of laser welding weld is generated;
S8, ABAQUS GUI interface is called through MATLAB to import INP file, the consistency of crystal plasticity finite element model with microstructure distribution of laser welding welds and the original laser welding weld microstructure distribution is verified;
S9, the METX toolbox is called by MATLAB to draw the polar figure of the EBSD data of the original laser welding weld and the polar figure of the grain set after noise reduction, and whether the grain set established is consistent with the original EBSD data is verified;
S10, the grain euler Angle is converted into radian value, and MATLAB is used to output the PYTHON file that assigns material properties to each grain, wherein the material property assignment code includes three main parts: automatically reading the grain Euler Angle radian value, converting the radian value to the Miller index, and assigning a grain size effect;
S11, ABAQUS is called through MATLAB to perform PYTHON for material property assignment, material properties are assigned to each grain, and the final INP file of the crystal plasticity finite element model for laser welding weld is generated;
S12, an empty dat file is generated by MATLAB to write the orientation information of each grain in the final crystal plasticity finite element model output by ABAQUS;
S13, ABAQUS is called through MATLAB to run the final generated INP file, and the subroutine of crystal plasticity finite element simulation is called to output the orientation information of each grain in the final generated crystal plasticity finite element model, METX is called to draw the polar figure of each grain in the final output, and is compared with the polar figure generated in step 8;
S14, the PYTHON file is written to extract the stress-strain curve of the crystal plasticity finite element simulation through MATLAB, and ABAQUS is called through MATLAB to execute PYTHON file to obtain the stress-strain curve of the crystal plasticity finite element simulation.

In the full-automatic full-process modeling method of the crystal plasticity finite element model for laser welding weld provided by the invention, the signal and data processing process include the following detailed steps:

### Step 1: EBSD Data Import and Data Set Creation

Import EBSD data: use MATLAB to call the METX toolbox to import EBSD (electronic backscatter diffraction) data for laser welding welds;

Create the data set: create the EBSD data set in the MATLAB workspace, which is the basis for subsequent processing.

### Step 2: Processing of Raw EBSD Data

Grain segmentation: the grains in the EBSD data are separated separately for separate processing.

Confidence index filtering: confidence index filtering is used to remove noise and untrusted parts of the data.

Small grain filtering: the grain in extremely small sizes is removed to reduce the impact of tiny components on the model.

Step 3: Extraction and Comparison of Data after Noise Reduction

Extract data: the key information is extracted from the EBSD data after noise reduction, such as the grain number, the equivalent diameter, the Euler Angle, the coordinates, and the like;

Draw grain distribution map: the grain distribution map after noise reduction is drawn and compared with the EBSD grain distribution map of the original laser welding weld.

### Step 4: EBSD Scan Data Processing

Extract dimension information: the length and width dimensions of the original EBSD scan data is obtained.

Calculate the step size: the step size of a single pixel point is calculated and the units are converted from microns (um) to millimeters (mm).

### Step 5: Enter the Model Meshing Parameters

Set the mesh size: the mesh size of the crystal plasticity finite element model of the laser welding weld is entered.

Define ABAQUS analysis step parameters: the initial analysis step, the minimum analysis step, and the maximum analysis step in an ABAQUS analysis step are set.

### Step 6: Automatic Creation of ABAQUS Initial Model

Writing Python scripts: Python scripts are written using MATLAB to create the initial model of ABAQUS.

Execute Python scripts: Python scripts are automatically executed to generate INP files for the initial model.

### Step 7: Node and Unit Data Processing

Read node and unit data: node and unit data of the initial model are read through MATLAB.

Generate grain collection data: according to the position of each grain and the pixel value occupied by each grain, a data file of the node and unit collection of each grain is generated in ABAQUS and is written into the INP file of the initial model.

### Step 8: Model Validation

Import INP file: ABAQUS GUI interface is called via MATLAB to import INP file including crystal plasticity finite element model with grain structure distribution.

Verify the consistency of the model: the consistency of the model and the original laser welding weld microstructure distribution is checked.

### Step 9: The Drawing and Verification of the Polar Figure

Drawing pole diagram: MATLAB is used to call the METX toolbox to draw the pole diagram of the original and de-noised grain sets.

Verify grain sets: the polar maps are compared to verify that the grain sets are consistent with the original EBSD data.

### Step 10: Grain Euler Angle Treatment and Material Properties

Convert Euler Angle: the Euler Angle of the grain is converted into the radian value.

Output material property assignment file: MATLAB is used to output Python files that assign material properties to each grain, including automatic reading, conversion, and size effect assignment of grain Euler angles.

### Step 11: Perform Material Property Assignment

Execute Python script: ABAQUS is called through MATLAB to execute Python script assigned by material properties, and material properties is assigned to each grain.

### Step 12: Data File Generation

Generate an empty dat file: for writing orientation information for each grain in the crystal plasticity finite element model of the ABAQUS output.

### Step 13: Simulation Run and Data Output

Run the INP file: ABAQUS is called through MATLAB to run the resulting INP file.

Output grain orientation information: the subroutine of crystal plastic finite element simulation is called, the orientation information of each grain is output, and a polar map is drawn for comparison.

### Step 14: Stress-Strain Curve Extraction

Write extraction script: the Python file to extract the stress-strain curve of the crystal plasticity finite element simulation is written through MATLAB.

Execute Python script: the Python file is executed by calling ABAQUS through MATLAB to obtain the stress-strain curve of crystal plasticity finite element simulation.

The data set provided by the invention includes important information such as a grain type, a grain ID, a grain orientation, a phase sequence, a grain rotation, and the like.

The formula of grain segmentation, confidence index filtering and small grain filtering provided by the invention is:

$$Δ θ = arccos ( \frac{trace ( {R}_{1}^{T} {R}_{2} ) - 1}{2} ) ;$$

wherein, R₁^{T} and R₂ respectively represent the rotation matrix of grains, and Δθ are the orientation angle difference between grains; If Δθ is greater than a threshold, the two grains are considered to belong to different grains;

$$f ( {CI}_{i} ) = \{ \begin{matrix}1 , & if {CI}_{i} > min Ci \\ 0 , & otherwise\end{matrix} ;$$

For each measurement point i, the confidence index is CIᵢ;

$$f ( g ) = \{ \begin{matrix}1 , & if size ( g ) > min Size \\ 0 , & otherwise\end{matrix} ;$$

wherein g is each grain and size(g) is a size of each grain;

The py file provided by the invention includes INP files for creating model entities, meshing, generating meshing elements, creating assemblies, establishing analysis steps, creating reference points, establishing reference point coupling constraints, adding fixed constraints, adding load constraints, and generating models;

The formulas for converting radian value into Miller index and grain size effect provided by the invention are respectively:

$$( \begin{matrix}u & r & h \\ v & s & k \\ w & t & l\end{matrix} ) = [ \text{} \begin{matrix}\begin{matrix}cos ψ cos φ - \\ sin ψ sin φ cos θ\end{matrix} & \begin{matrix}sin ψ cos θ + \\ cos ψ sin φ cos θ\end{matrix} & sin φ sin θ \\ \begin{matrix}- cos ψ sin φ - \\ sin ψ cos φ cos θ\end{matrix} & \begin{matrix}- sin ψ sin φ + \\ cos ψ cos φ cos θ\end{matrix} & cos φ sin θ \\ sin ψ sin θ & - cos ψ sin θ & cos θ\end{matrix} ] ;$$

$${τ}_{i} = {τ}_{0} + {Kd}^{- 0.5} ( i = 1 , 2 , 3 … ) ;$$

wherein θ is the nutation angle, ψ is the precession angle, and φ is the rotation angle; u, v, w are crystal direction indices; h, k, l are crystal face indices; i is the number of the grain, T0 is the initial yield strength of the grain, K is the influence coefficient of the reaction grain boundary on the deformation, and d is the equivalent diameter of the grain.

The invention relates to an full-automatic full-process modeling method of crystal plasticity finite element model for laser welding weld, which is characterized by: based on the EBSD data of aluminum alloy laser welding welds, MATLAB is used to call ABAQUS and PYTHON for co-simulation programming to realize the full-automatic, full-process high-precision and high-efficiency modeling of the finite element model of the microstructure of aluminum alloy laser welding welds, including the following steps:
Step 1: MATLAB is used to call METX toolbox to import EBSD data of laser welding weld of aluminum alloy, and create EBSD data set in MATLAB working area, which includes important information such as a grain type, a grain ID, a grain orientation, a phase sequence and a grain rotation.
Step 2: Grain segmentation, confidence index filtering, small grain filtering and other methods are used to establish a grain set of the original EBSD data, delete the grain in a very small size, and reduce the noise of the original laser welding weld EBSD data to improve the quality of EBSD data, wherein the formula of grain segmentation, confidence index filtering and small grain filtering is as follows:

$$Δθ = arccos ( \frac{trace ( {R}_{1}^{T} {R}_{2} ) - 1}{2} ) ;$$

wherein, R₁^{T} and R₂ respectively represent the rotation matrix of grains, and Δθ are the orientation angle difference between grains; if Δθ is greater than a threshold, the two grains are considered to belong to different grains;

$$f ( {CI}_{i} ) = \{ \begin{matrix}1 , & if {CI}_{i} > min Ci \\ 0 , & otherwise\end{matrix} ;$$

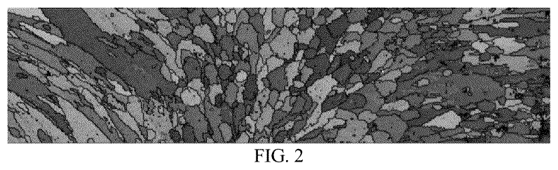

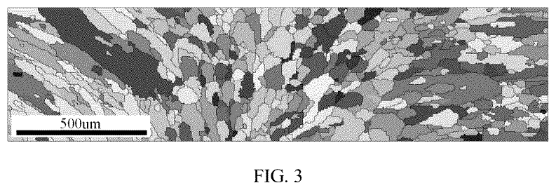

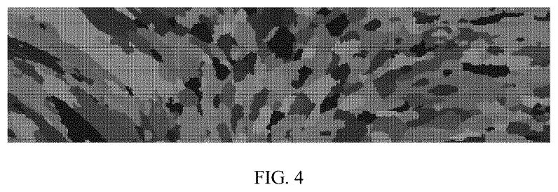

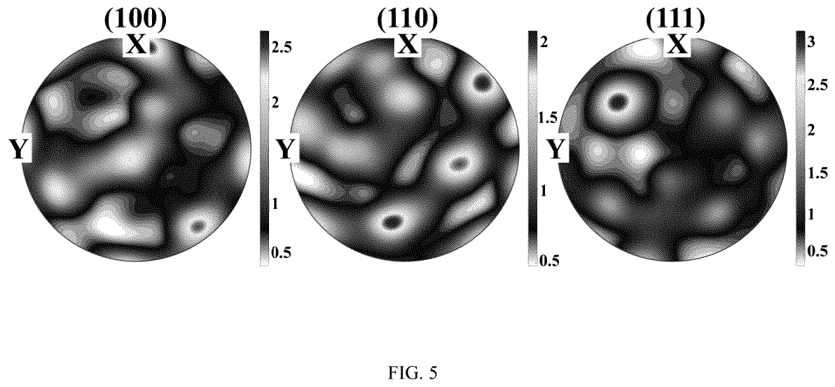

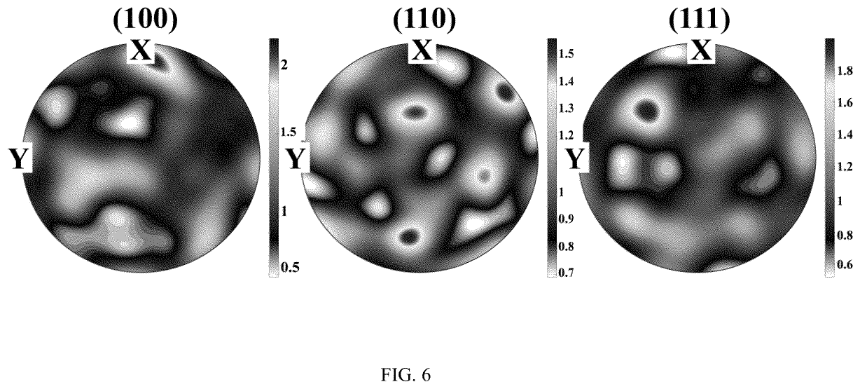

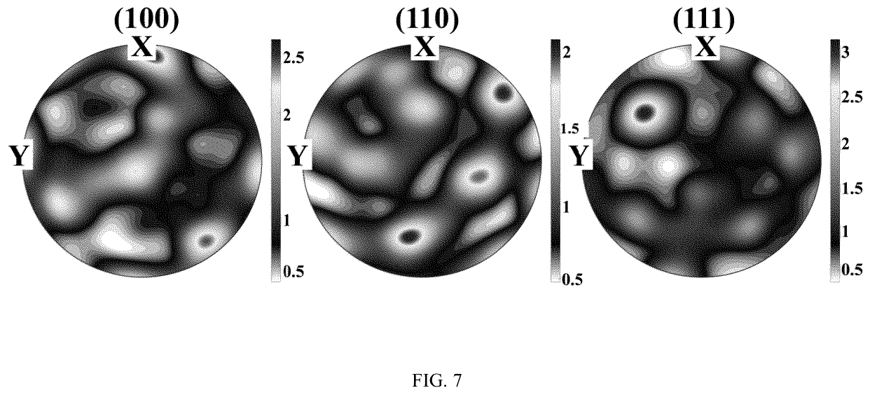

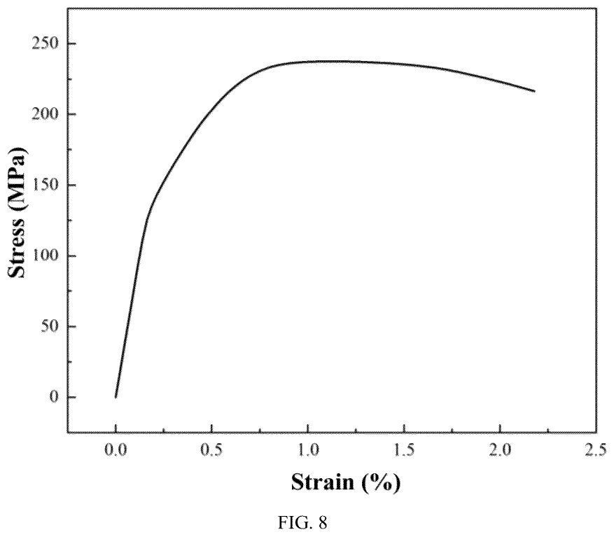

For each measurement point i, the confidence index is CIᵢ;

$$f ( g ) = \{ \begin{matrix}1 , & if size ( g ) > min Size \\ 0 , & otherwise\end{matrix} ;$$

wherein g is each grain and size(g) is the size of each grain.
Step 3: the EBSD data after noise reduction is extracted, including the grain number, the equivalent diameter, the euler angle of grain orientation, the grain pixel coordinates and the grain set of each grain, the grain distribution map after noise reduction is drawn, and is compared with the EBSD grain distribution map of the original laser welding weld, as shown in FIG. 2 and FIG. 3 respectively.
Step 4: the length and width of the original EBSD scan data is extracted through MATLAB, the step size of a single pixel of the EBSD scan data of the aluminum alloy laser welding weld is calculated, and the unit is converted from um to mm.
Step 5: the mesh size of the aluminum alloy laser welding weld crystal plastic finite element model, the initial analysis step length, the minimum analysis step length and the maximum analysis step length in the ABAQUS analysis step are entered.
Step 6: the py files are written and generated to create the initial model of ABAQUS through MATLAB, including INP files of creating model entities, meshing, generating meshing elements, creating assemblies, establishing analysis steps, creating reference points, establishing reference point coupling constraints, adding fixed constraints, adding load constraints, and generating models, wherein the py file is executed by calling PYTHON through MATLAB to automatically generate the INP file of the initial model.
Step 7: each node data and unit data of the initial model are read by MATLAB, and a data file of the node and unit set of each grain in ABAQUS is generated according to the location (X and Y coordinates) of each grain and the pixel value occupied by each grain, and then written into the INP file of the initial model, wherein the INP file of the finite element model of crystal plasticity including the grain structure distribution of laser welding welds is generated.
Step 8: ABAQUS GUI interface is called through MATLAB to import INP file to verify the consistency between the crystal plastic finite element model with laser welding weld grain structure distribution and the original laser welding weld microstructure distribution, as shown in FIG. 4.
Step 9: METX toolbox is called by MATLAB to draw the polar figure of the EBSD data of the original laser welding weld and the polar figure of the grain set after noise reduction processing, to verify whether the grain set established is consistent with the original EBSD data, as shown in FIG. 5 and FIG. 6 respectively.
Step 10: the grain Euler Angle is converted into the radian value, and MATLAB is used to output the PYTHON file that assigns material properties to each grain. The material property assigning code includes three main parts: automatically reading the grain Euler angle radian value, converting the radian value to Miller index, and assigning the grain size effect. The formulas for converting the radian value to Miller index and assigning the grain size effect are as follows:

$$( \begin{matrix}u & r & h \\ v & s & k \\ w & t & l\end{matrix} ) = [ \text{} \begin{matrix}\begin{matrix}cos ψ cos φ - \\ sin ψ sin φ cos θ\end{matrix} & \begin{matrix}sin ψ cos θ + \\ cos ψ sin φ cos θ\end{matrix} & sin φ sin θ \\ \begin{matrix}- cos ψ sin φ - \\ sin ψ cos φ cos θ\end{matrix} & \begin{matrix}- sin ψ sin φ + \\ cos ψ cos φ cos θ\end{matrix} & cos φ sin θ \\ sin ψ sin θ & - cos ψ sin θ & cos θ\end{matrix} ] ;$$

$${τ}_{i} = {τ}_{0} + {Kd}^{- 0.5} ( i = 1 , 2 , 3 … ) ;$$

wherein θ is the nutation angle, ψ is the precession angle, and φ is the rotation angle; u, v, w are crystal direction indices; h, k, l are crystal face indices; i is the number of the grain, T0 is the initial yield strength of the grain, K is the influence coefficient of the reaction grain boundary on the deformation, and d is the equivalent diameter of the grain;
Step 11: ABAQUS is called through MATLAB to perform PYTHON for material property assignment, assign material properties to each grain, and generate the final INP file of the crystal plasticity finite element model for the laser welding weld.
Step 12: an empty dat file is generated through MATLAB to write the orientation information of each grain in the final generated crystal plasticity finite element model output by ABAQUS.
Step 13: ABAQUS is called through MATLAB to run the final generated INP file, and call the subroutine of crystal plasticity finite element simulation to output the orientation information of each grain in the final generated crystal plasticity finite element model; METX is called to draw the polar figure of each grain in the final output, and is compared with the polar figure generated in step 8, as shown in FIG. 7.
Step 14: the PYTHON file is written through MATLAB to extract the stress-strain curve of the crystal plasticity finite element simulation, and ABAQUS is called through MATLAB to execute the PYTHON file to obtain the stress-strain curve of the crystal plasticity finite element simulation, as shown in FIG. 8.

The invention adopts MATLAB to call ABAQUS and PYTHON to carry out joint simulation programming, proposes a full-process full-automatic modeling method of the crystal plasticity finite element model for laser welding weld, and realizes the one-key generation of the crystal plasticity finite element model for laser welding weld based on EBSD experimental data. In addition, the full-process full-automatic modeling method for laser welding weld crystal plasticity finite element is universal model, and can be used for the establishment of the finite element model of the microstructure crystal plasticity of the base material of aluminum alloy, titanium alloy, magnesium alloy, stainless steel and other materials.

It should be noted that embodiments of the invention can be realized by hardware, software, or a combination of software and hardware. The hardware part can be realized by using special logic. The software portion can be stored in the memory and executed by an appropriate instruction execution system, such as a microprocessor or specially designed hardware. A person of ordinary skill in the art may understand that the above devices and methods may be implemented using computer-executable instructions and/or contained in processor control code, Such code is provided, for example, on a carrier medium such as a disk, CD or DVD-ROM, on a programmable memory such as read-only memory (firmware), or on a data carrier such as an optical or electronic signal carrier. The device and its module of the invention can be realized by hardware circuits of programmable hardware devices such as VLics or gate arrays, semiconductors such as logic chips, transistors, and the like, or by software executed by various types of processors. It can also be achieved by a combination of the above hardware circuits and software, such as firmware.

The invention adopts MATLAB to call ABAQUS and PYTHON to carry out joint simulation programming, proposes a full-process full-automatic modeling method of the crystal plasticity finite element model for laser welding weld, and realizes the one-key generation of the crystal plasticity finite element model for laser welding weld based on EBSD experimental data. In addition, the full-process full-automatic crystal plasticity finite element modeling method for laser welding weld is universal, and can be used for the establishment of the finite element model for the microstructure crystal plasticity of the base material of aluminum alloy, titanium alloy, magnesium alloy, stainless steel and other materials.

The above is only the specific embodiment of the invention, but the scope of protection of the invention is not limited to this, and any modification, equivalent replacement and improvement made by those skilled in the art within the technical scope disclosed by the invention and within the spirit and principles of the invention shall be covered by the scope of protection of the invention.

What is claimed is:

1. A full-automatic full-process modeling method of the crystal plasticity finite element model for laser welding weld, characterized by combining MATLAB software and ABAQUS finite element analysis software, using METX toolbox to process EBSD microstructure data of laser welding welds, and constructing and verifying the crystal plasticity finite element model; Firstly, EBSD data is denoised, and then grain information is extracted for model building, then, writing and executing scripts in MATLAB to automatically create ABAQUS model, importing grain data to generate INP file, and assigning material properties, Finally, outputting grain orientation information is by running a simulation, and extracting a stress-strain curve of simulation results, the full-process is fully automated from the EBSD data to crystal plasticity finite element model;
which comprises the following steps:
Step 1: using MATLAB to call METX toolbox to import EBSD data for laser welding welds, and creating EBSD data set in MATLAB workspace;
Step 2: using grain segmentation, confidence index filtering, small grain filtering and other methods to establish a grain set of original EBSD data, removing a grain in a very small size, and reduce a noise of original laser welding weld EBSD data;
Step 3: extracting the EBSD data after noise reduction, comprising a grain number, an equivalent diameter, an euler angle of grain orientation, grain pixel coordinates, a grain set and other data of each grain, drawing a grain distribution map after noise reduction, and comparing with original laser welding weld EBSD grain distribution map;
Step 4: extracting a length and a width of original EBSD scan data through MATLAB, calculating a step size of a single pixel of EBSD scan data for laser welding welds, and converting a unit from um to mm;
Step 5: entering a mesh size of crystal plasticity finite element model of the laser welding weld, an initial analysis step length, the minimum analysis step length and the maximum analysis step length in ABAQUS analysis step;
Step 6: writing and generating py files to create an initial model of ABAQUS through MATLAB; executing the py file by calling PYTHON through MATLAB to automatically generate the INP file of the initial model;
Step 7: reading each node data and unit data of the initial model through MATLAB, and generating a data file of a node and a unit set of each grain in ABAQUS according to a location of each grain and a pixel value occupied by each grain, and writing into the INP file of the initial model, generating the INP file of the crystal plasticity finite element model, including the grain structure distribution of laser welding weld;
Step 8: calling ABAQUS GUI interface through MATLAB to import the INP file to verify a consistency of crystal plasticity finite element model with grain structure distribution of laser welding weld and original laser welding weld microstructure distribution;
The pixel gray level modulation structure based on phase change materials, is characterized by comprising n multi-layer phase change unit arrays in each sub-pixel, an upper all-dielectric filter structure, an all-dielectric intermediate cavity, a lower all-dielectric filter structure and a crossbar control structure;
Step 9: using MATLAB to call METX toolbox to draw a polar figure of the EBSD data of the original laser welding weld and a polar figure of the grain set after noise reduction, and verifying whether the grain set established is consistent with the original EBSD data;
Step 10: converting a grain Euler angle into a radian value, and using MATLAB to output the PYTHON file that assigns material properties to each grain, wherein the material property assigning code comprises three main parts: automatically reading a grain Euler angle radian value, converting radian value to Miller index, and assigning a grain size effect;
Step 11: using MATLAB to call ABAQUS to perform PYTHON for material property assignment, assigning material properties to each grain, and generating the INP file of the crystal plasticity finite element model, including the grain structure distribution of laser welding weld;
Step 12: generating an empty dat file through MATLAB to write an orientation information of each grain in the final crystal plasticity finite element model output by ABAQUS;
Step 13: calling ABAQUS through MATLAB to run the final generated INP file, and calling a subroutine of crystal plasticity finite element simulation to output the orientation information of each grain in the final generated crystal plasticity finite element model; calling METX to draw a polar figure of each grain in the final output, and comparing with the polar figure generated in step 8;
Step 14: writing the PYTHON file to extract a stress-strain curve of crystal plasticity finite element simulation through MATLAB, and calling ABAQUS to execute the PYTHON file through MATLAB to obtain the stress-strain curve of crystal plasticity finite element simulation;
wherein the data set comprises important information such as a grain type, a grain ID, a grain orientation, a phase sequence, and a grain rotation.

2. The full-automatic full-process modeling method of the crystal plasticity finite element model for laser welding weld according to claim 1, characterized in that a formula for grain segmentation, confidence index filtering, small grain filtering is:
$$Δθ = arccos ( \frac{trace ( {R}_{1}^{T} {R}_{2} ) - 1}{2} ) ;$$
wherein a grain rotation is expressed as R1 and R2, and Δθ is an orientation angle difference between two points; If Δθ>angle, two points are considered to belong to different grains;
$$f ( {CI}_{i} ) = \{ \begin{matrix}1 , & if {CI}_{i} > min Ci \\ 0 , & otherwise\end{matrix} ;$$
for each measurement point i, the confidence index is CIᵢ;
$$f ( g ) = \{ \begin{matrix}1 , & if size ( g ) > min Size \\ 0 , & otherwise\end{matrix} ;$$
wherein g is each grain and size(g) is a size of each grain.

3. The full-automatic full-process modeling method of the crystal plasticity finite element model for laser welding weld according to claim 1, characterized in that the py file comprises the INP files for creating model entities, meshing, generating meshing elements, creating assemblies, establishing analysis steps, creating reference points, establishing reference point coupling constraints, adding fixed constraints, adding load constraints, and generating model.

4. The full-automatic full-process modeling method of the crystal plasticity finite element model for laser welding weld according to claim 1, characterized in that the formula that the radian value is converted to the Miller index and the grain size effect are as follows:
$$( \begin{matrix}u & r & h \\ v & s & k \\ w & t & l\end{matrix} ) = [ \text{} \begin{matrix}\begin{matrix}cos ψ cos φ - \\ sin ψ sin φ cos θ\end{matrix} & \begin{matrix}sin ψ cos θ + \\ cos ψ sin φ cos θ\end{matrix} & sin φ sin θ \\ \begin{matrix}- cos ψ sin φ - \\ sin ψ cos φ cos θ\end{matrix} & \begin{matrix}- sin ψ sin φ + \\ cos ψ cos φ cos θ\end{matrix} & cos φ sin θ \\ sin ψ sin θ & - cos ψ sin θ & cos θ\end{matrix} ] ;$$
$${τ}_{i} = {τ}_{0} + {Kd}^{- 0.5} ( i = 1 , 2 , 3 … ) ;$$
herein θ is a nutation angle, ψ is a precession angle, and φ is a rotation angle; u, v, w are crystal direction indices; h, k, l are crystal face indices; i is the number of the grain, T0 is an initial yield strength of the grain, K is an influence coefficient of a reaction grain boundary on a deformation, and d is an equivalent diameter of the grain.

5. The full-automatic full-process modeling system of the crystal plasticity finite element model for laser welding weld as the described method according to claim 1, characterized by comprising:
data processing module, equipped with MATLAB software and METX toolbox, used to import and process EBSD data of laser welding welds, comprising grain segmentation, confidence index filtering and small grain filtering;
model building module, used to extract and process data through MATLAB, comprising calculating an individual pixel step size of EBSD scanning data, converting units, and generating a grain distribution map;
finite element analysis module, using ABAQUS software and combining data generated by MATLAB, used to create an initial model, a grain node and an unit collection data file, and generating the INP file of the crystal plasticity finite element model, including the grain structure distribution of laser welding weld;
model verification module, using ABAQUS GUI and MATLAB combined with METX toolbox, used to import models, verify a consistency of models, draw and compare polar figures;
material property assigning module, used to call ABAQUS through MATLAB to execute Python script, assign material properties to each grain, and generate the final INP file of the finite element model;
data output module, used to generate and process ABAQUS output data, comprising orientation information and stress-strain curves for each grain in the crystal plasticity finite element model.
